(19) United States
(12) Patent Application Publication
(10) Pub. No.: US 2017/0333571 A1
(43) Pub. Date: Nov. 23, 2017

(54) Targeted Delivery Platform for Delivery of Therapeutics

(71) Applicant: The Regents of the University of California, Oakland, CA (US)

(72) Inventors: Aditi Bhargava, Hillsborough, CA (US); Peter T. Ohara, San Francisco, CA (US); Luc Jasmin, Los Angeles, CA (US)

(21) Appl. No.: 15/522,731

(22) PCT Filed: Nov. 3, 2015
(86) PCT No.: PCT/US15/58845

§ 371 Date: Apr. 27, 2017

Related U.S. Application Data
(60) Provisional application No. 62/075,042, filed on Nov. 4, 2014.

(51) Int. Cl. A61K 47/69; A61K 47/68; A61K 31/7052; C07K 16/24; A61K 47/64; A61K 38/17; A61K 47/60

(52) U.S. Cl. CPC A61K 47/6929; A61K 38/1709; A61K 47/6845; A61K 47/60; C07K 16/241; A61K 47/64; A61K 31/7052; A61K 47/6415; C07K 2317/21; C07K 2317/24; C07K 2317/76

(57) ABSTRACT

The present disclosure provides a targeted delivery platform for delivery of a therapeutic molecule to a target cell. The targeted delivery platform includes a complex that includes the therapeutic molecule, a polyalkylene glycol polymer and a targeting protein. Also provided herein are methods of making the complex and methods of using the complex to deliver the therapeutic molecule to a target cell to achieve treatment of a disease.

### CROSS-REFERENCE TO RELATED APPLICATION

This application is based upon and claims priority to U.S. provisional patent application 62/075,042, filed Nov. 4, 2014, which is incorporated herein by reference in its entirety.

### STATEMENT REGARDING FEDERALLY SPONSORED RESEARCH

This invention was made with government support under Grant No. DK080787 awarded by the National Institutes of Health. The government has certain rights in the invention.

### INTRODUCTION

Targeted delivery of a therapeutic molecule to a target cell is highly desirable due to the associated advantages. For example, if a therapeutic molecule can be targeted to a particular cell where the activity of the therapeutic molecule is needed, a lower amount of the therapeutic molecule would be efficacious, lowering the cost of the treatment. The targeted delivery of the therapeutic molecule as well as the lower dose of the therapeutic molecule would also reduce side effects that result from off target effect therapeutic molecule as well as immune response to the therapeutic molecule.

Although targeted delivery of therapeutic molecule has been attempted, there is a need for specific complexes that provide effective delivery of the therapeutic molecule.

### SUMMARY

The present disclosure provides a targeted delivery platform for delivery of a therapeutic molecule to a target cell. The targeted delivery platform includes a complex that includes the therapeutic agent, a polyalkylene glycol polymer and a targeting protein. Also provided herein are methods of making the complex and methods of using the complex to deliver the therapeutic agent to a target cell to achieve treatment of a disease.

### BRIEF DESCRIPTION OF THE DRAWINGS

FIGS. 1A-1C. FIG. 1A provides a schematic of the Cholera toxin subunits A and B. FIG. 1B is a schematic of a therapeutic drug or antibody PEG-linker complex encapsulated in Cholera toxin subunit B (CTB) pentamer. FIG. 1C depicts a model for targeted delivery of therapeutic drug or antibody PEG-linker complex via CTB pentamer (Ab-CQ) to an intestinal M cell or enterocyte upon administration of Ab-CQ to lumen of the colon.

FIG. 2 shows the multimers of CTB, CTB conjugated to PEG-Mal, and CTB conjugated to SMCC.

FIGS. 3A-3C depicts colon of rats treated with CQ, CQ-anti-TNF-α, or anti-TNF-α.

FIGS. 4A-4F depicts9 results from analysis of colon of rats treated with CQ, CQ-anti-TNF-α or anti-TNF-α.

FIG. 5 shows results of detection of anti-TNF-α antibody in serum of mice administered CQ-anti-TNF-α or anti-TNF-α via the indicated routes of administration.

FIGS. 6A-6B show results from analysis of colon of Crohn's colitis mice models treated with CQ, CQ-anti-TNF-α or anti-TNF-α via the indicated routes of administration.

FIGS. 7A-7D show colon of mice treated with CQ, CQ-Humira or Humira.

FIGS. 8A-8E shows results from analysis of colon of Crohn's colitis mice models treated with CQ, CQ-Humira or Humira.

FIGS. 9A-9D provides a higher magnification image of cross section of colon of Crohn's colitis mice models treated with CQ, CQ-Humira or Humira.

FIGS. 10A and 10B depict results from analysis of colon of mice treated with CQ, CQ-Humira, or Humira.

FIG. 11A-11D shows colon from mice with Crohn's colitis treated with Remicade-CQ and controls.

FIG. 12A-12C show results from analysis of colon of Crohn's colitis mice models treated with Urocortin-1.

FIG. 13A-13C show analysis of CQ-dsRNA complex.

FIG. 14 show analysis of CQ-antibody complex.

FIG. 15 graphically describes the immogenicity studies for CQ.

FIG. 16 is a graph showing IgG (using ELISA) concentration in serum from uninjected naïve mice, in pre-immune serum, after single, and repeated doses of CQ.

### DEFINITIONS

The terms “patient” or “subject” are used interchangeably to refer to a human or a non-human animal (e.g., a mammal). Exemplary subjects may include cattle, dogs, cats, guinea pigs, rabbits, rats, mice, horses, and so on.

The terms “treat”, “treating”, treatment” and the like refer to a course of action (such as administering a complex as disclosed herein) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject.

The term “in need of treatment” as used herein refers to a judgment made by a physician or other caregiver that a subject requires or will benefit from treatment. This judgment is made based on a variety of factors that are in the realm of the physician's or caregiver's expertise.

The terms “prevent”, “preventing”, “prevention” and the like refer to a course of action (such as administering a complex disclosed herein) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state.

The term “in need of prevention” as used herein refers to a judgment made by a physician or other caregiver that a subject requires or will benefit from preventative care. This judgment is made based on a variety of factors that are in the realm of a physician's or caregiver's expertise.

The phrase “therapeutically effective amount” refers to the administration of a complex as disclosed herein to a subject, either alone or as a part of a pharmaceutical composition and either in a single dose or as part of a series of doses, in an amount that is capable of having any detectable, positive effect on any symptom, aspect, or characteristics of a disease, disorder or condition when administered to a patient. The therapeutically effective amount can be ascertained by measuring relevant physiological effects.

The phrase “in a sufficient amount to effect a change” means that there is a detectable difference between a level of an indicator measured before (e.g., a baseline level) and after administration of a particular therapy. Indicators include any objective parameter (e.g., tissue histology) or subjective parameter (e.g., a subject's feeling of well-being).

As used herein, the term “variant” in the context of a nucleic acid sequence or a protein sequence encompasses naturally-occurring variants (e.g., homologs and allelic variants) and non-naturally-occurring variants (e.g., recombinantly modified). Naturally-occurring variants include homologs, i.e., nucleic acids and polypeptides that differ in nucleotide or amino acid sequence, respectively, from one species to another. Naturally-occurring variants include allelic variants, i.e., nucleic acids and polypeptides that differ in nucleotide or amino acid sequence, respectively, from one individual to another within a species. Non-naturally-occurring variants include nucleic acids and polypeptides that comprise a change in nucleotide or amino acid sequence, respectively, where the change in sequence is artificially introduced, e.g., the change is generated in the laboratory or other facility by human intervention (“hand of man”).

The terms “antibodies” (Abs) and “immunoglobulins” (Igs) refer to glycoproteins having the same structural characteristics. While antibodies exhibit binding specificity to a specific antigen, immunoglobulins include both antibodies and other antibody-like molecules which lack antigen specificity. Antibodies are described in detail hereafter.

The term “monoclonal antibody” refers to an antibody obtained from a population of substantially homogeneous antibodies, that is, the individual antibodies comprising the population are identical except for possible naturally occurring mutations that may be present in minor amounts. Monoclonal antibodies are highly specific, being directed against a single antigenic site. In contrast to polyclonal antibody preparations, which can include different antibodies directed against different determinants (epitopes), each monoclonal antibody is directed against a single determinant on the antigen.

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the term are Fab′, Fv, F(ab′)₂, and or other antibody fragments that retain specific binding to antigen, and monoclonal antibodies. An antibody may be monovalent or bivalent.

Antibodies may exist in a variety of other forms including, for example, Fv, Fab, and (Fab)₂, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986).

Chimeric antibodies are antibodies whose light and heavy chain genes have been constructed, typically by genetic engineering, from antibody variable and constant region genes belonging to different species. For example, the variable segments of the genes from a non-human monoclonal antibody may be joined to human constant segments, such as gamma 1 and gamma 3. An example of a therapeutic chimeric antibody is a hybrid protein composed of the variable or antigen-binding domain from a rabbit antibody and the constant or effector domain from a human antibody (e.g., the anti-Tac chimeric antibody made by the cells of A.T.C.C. deposit Accession No. CRL 9688), although other mammalian species may be used.

As used herein, the term “humanized antibody” or “humanized immunoglobulin” refers to an non-human (e.g., mouse or rabbit) antibody containing one or more amino acids that have been substituted with a correspondingly positioned amino acid from a human antibody. In some cases, humanized antibodies produce a reduced immune response in a human host, as compared to a non-humanized version of the same antibody.

In the context of an antibody, the term “isolated” refers to an antibody that has been separated and/or recovered from contaminant components of its natural environment; such contaminant components include materials which might interfere with diagnostic or therapeutic uses for the antibody, and may include enzymes, hormones, and other proteinaceous or nonproteinaceous solutes.

The term “specific binding” refers to the ability of a targeting protein to preferentially bind to a particular moiety that is present on a corresponding target cell while not binding to a significant extent to a cell not having the particular moiety. In certain embodiments, a specific binding interaction will discriminate between target cell and a non-target cell in a sample, in some embodiments more than about 10 to 100-fold or more (e.g., more than about 1000- or 10,000-fold).

The term “specific binding” in the context of an antibody refers to the ability of an antibody to preferentially bind to a particular analyte that is present in a homogeneous mixture of different analytes. In certain embodiments, a specific binding interaction will discriminate between desirable and undesirable analytes in a sample, in some embodiments more than about 10 to 100-fold or more (e.g., more than about 1000- or 10,000-fold).

The terms “polypeptide” and “protein”, used interchangeably herein, refer to a polymeric form of amino acids of length more than about 50 amino acids, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like. The term “peptide” refers to polypeptides that are 8-50 residues (e.g., 8-20 residues) in length.

As used herein the term “isolated” is meant to describe a compound of interest (e.g., either a polynucleotide or a polypeptide) that is in an environment different from that in which the compound might naturally occur.

“Purified” as used herein refers to a complex removed from an environment in which it was produced and is at least 60% free, preferably 75% free, and most preferably 90% free from other components with which it is naturally associated or with which it was otherwise associated with during production.

The term “gene” as used herein includes sequences of nucleic acids that when present in an appropriate host cell facilitates production of a gene product. “Genes” can include nucleic acid sequences that encode proteins, and sequences that do not encode proteins, and includes genes that are endogenous to a host cell or are completely or partially recombinant (e.g., due to introduction of a exogenous polynucleotide encoding a promoter and a coding sequence, or introduction of a heterologous promoter adjacent an endogenous coding sequence, into a host cell). For example, the term “gene” includes nucleic acid that can be composed of exons and introns. Sequences that code for proteins are, for example, sequences that are contained within exons in an open reading frame between a start codon and a stop codon., “Gene” as used herein can refer to a nucleic acid that includes, for example, regulatory sequences such as promoters, enhancers and all other sequences known in the art that control the transcription, expression, or activity of another gene, whether the other gene comprises coding sequences or non-coding sequences. In one context, for example, “gene” may be used to describe a functional nucleic acid comprising regulatory sequences such as promoter or enhancer. The expression of a recombinant gene may be controlled by one or more heterologous regulatory sequences. “Heterologous” refers to two elements that are not normally associated in nature.

The term “polynucleotide” refers to polymers of nucleotides, and includes but is not limited to single stranded or double stranded molecule of DNA, RNA, or DNA/RNA hybrids including polynucleotide chains of regularly and irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides wherein the substitution or attachment of various entities or moieties to the nucleotide units at any position, as well as naturally-occurring or non-naturally occurring backbones, are included.

The term “polyribonucleotide” refers to a polynucleotide comprising two or more modified or unmodified ribonucleotides and/or their analogs.

The term “ribonucleotide” and the phrase “ribonucleic acid” (RNA), refer to a naturally occurring or non-naturally occurring (artificial, synthetic), modified or unmodified nucleotide or polynucleotide. A ribonucleotide unit comprises an oxygen attached to the 2′ position of a ribosyl moiety that has a nitrogenous base attached in N-glycosidic linkage at the 1′ position of a ribosyl moiety, and a moiety that either allows for linkage to another nucleotide or precludes linkage. “Ribonucleic acid” as used herein can have a naturally occurring or modified phosphate backbone (e.g., as produced by synthetic techniques), and can include naturally-occurring or non-naturally-occurring, genetically encodable or non-genetically encodable, residues.

The term “deoxyribonucleotide” refers to a nucleotide or polynucleotide lacking an OH group at the 2′ and/or 3′ position of a sugar moiety. Instead it has a hydrogen bonded to the 2′ and/or 3′ carbon. “Deoxyribonucleic acid” as used herein can have a naturally occurring or modified phosphate backbone (e.g., as produced by synthetic techniques), and can include naturally-occurring or non-naturally-occurring, genetically encodable or non-genetically encodable, residues.

The phrase “RNA interference” and the term “RNAi” refer to the process by which a polynucleotide or double stranded polynucleotide comprising at least one ribonucleotide unit exerts an effect on a biological process through disruption of gene expression. The process includes but is not limited to gene silencing by degrading mRNA, interactions with tRNA, rRNA, hnRNA, cDNA and genomic DNA, as well as methylation of DNA and ancillary proteins.

The term “siRNA” and the phrase “short interfering RNA” refer to a double stranded nucleic acid that is capable of performing RNAi and that is between 18 and 30 base pairs in length (i.e., a duplex region of between 18 and 30 base pairs). Additionally, the term siRNA and the phrase “short interfering RNA” include nucleic acids that also contain moieties other than ribonucleotide moieties, including, but not limited to, modified nucleotides, modified internucleotide linkages, non-nucleotides, deoxynucleotides and analogs of the aforementioned nucleotides.

siRNAs can be duplexes, and can also comprise short hairpin RNAs, RNAs with loops as long as, for example, 4 to 23 or more nucleotides, RNAs with stem loop bulges, micro-RNAs, and short temporal RNAs. RNAs having loops or hairpin loops can include structures where the loops are connected to the stem by linkers such as flexible linkers. Flexible linkers can be comprised of a wide variety of chemical structures, as long as they are of sufficient length and materials to enable effective intramolecular hybridization of the stem elements. Typically, the length to be spanned is at least about 10-24 atoms. In exemplary embodiments, siRNA have a duplex region of 18-21 bp long.

The phrase “long double stranded RNA” or “long dsRNA” refers to a double stranded nucleic acid that is capable of performing RNAi and that is between 100 base pairs to 1500 base pairs in length (i.e., a duplex region of between 100 base pairs to 1500 base pairs). The RNA includes a sequence substantially complementary to an mRNA sequence of a target eukaryotic gene or viral gene. The double-strand region of the RNA is at least 100 bp, 200 bp, 400 bp, 1000 bp or 1500 bp in length, and, no more than 2000 bp in length.

As used herein, the phrase “microRNA” (also referred to herein interchangeably as “miRNA” or “miR”) refers to a microRNA (miRNA) molecule acting as a post-transcriptional regulator. Typically, the miRNA molecules are RNA molecules of about 20 to 22 nucleotides in length which can be loaded into a RISC complex and which direct the cleavage of another RNA molecule, wherein the other RNA molecule comprises a nucleotide sequence essentially complementary to the nucleotide sequence of the miRNA molecule.

The phrase “mammalian cell” refers to a cell of any mammal, including humans. The phrase refers to cells in vivo, such as, for example, in an organism or in an organ of an organism. The phrase also refers to cells in vitro, such as, for example, cells maintained in cell culture.

As used herein, the term “about” in the context of a value or range refers to values or ranges that are within ±15% of the disclosed value or range.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a nanoparticle” includes a plurality of such nanoparticles and reference to “the complex” includes reference to one or more complexes and equivalents thereof, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a targeted delivery platform for delivery of a therapeutic molecule to a target cell. The targeted delivery platform includes a complex that includes the therapeutic molecule, a polyalkylene glycol polymer and a targeting protein. Also provided herein are methods of making the complex and methods of using the complex to deliver the therapeutic molecule to a target cell to achieve treatment of a disease.

### Complexes of Therapeutic Molecules

A complex for targeted delivery of a therapeutic molecule to a target mammalian cell is disclosed. The complex may include a targeting protein that encapsulates nanoparticles of a therapeutic agent. The nanoparticles may include the therapeutic agent and polyalkylene glycol. Polyalkylene glycol may include a linker moiety that may facilitate covalent attachment of the nanoparticles to the targeting protein.

As described in the Examples section of the application, generating a nanoparticle of a therapeutic molecule and polyalkylene glycol-linker moiety and encapsulating the nanoparticles in a targeting protein results in formation of a complex that effectively delivers the therapeutic molecule to a target cell.

### Targeting Protein

The targeting protein to be used for encapsulating a therapeutic agent, for example, as present in a nanoparticle, may be selected on the basis of the target cell to which the therapeutic molecule is to be delivered.

The target cell may be selected based on the tissue afflicted with the disease and where the treatment, e.g., by delivering the therapeutic agent, is to be targeted. For example, for treatment of a cancer, the target cell may be the cancer cell; for treatment of inflammatory bowel disease, the target cell may be an intestinal cell; for treatment of spinal pain, the target cell may be a neuron, and so on. In certain cases, the target cell may be one or more of an intestinal cell, a neuron, a retinal cell, a muscle cell, a pancreatic cell, a liver cell, a kidney cell, blood cell, or the like. Numerous examples of targeting proteins that specifically bind to a target mammalian cell are known. Exemplary targeting proteins include proteins from bacteria, fungi and plants. In certain cases, a targeting protein may be a protein from a pathogenic bacteria, where the bacterial protein is involved in delivering the bacteria or a component (e.g., a pathogenic component) thereof into a mammalian cell. Bacterial proteins that may be used as the targeting protein in the complexes described herein may be bacterial proteins that bind to a cognate molecule on cell surface of a target cell and enter the cell by a number of mechanisms, such as, formation of a channel in the cell membrane and/or internalization by the cell by endocytosis, for example.

In certain cases, the targeting protein may be a bacterial toxin or a variant thereof. Exemplary bacterial toxins are listed in Table 1 below. It is noted that although the toxin originate in a bacteria, the toxin need not be isolated from the bacteria but can be generated by recombinant means, such as by expression in a host genetically modified to express the toxin or by chemical synthesis.

TABLE 1
Bacterial toxins and target mammalian cell

| BACTERIA | TOXIN | TARGET | TARGET MECHANISM |
|---|---|---|---|
| Clostridium | Botulinum | neurons | C-terminal region of Heavy chain binds to |
| botulinum | neurotoxin | | surface of target nerve cells |
| Bacillus | Anthrax | Heart, muscle and | Enters cell via ANTXR1 and ANTR2 |
| anthracis | | liver | receptors |
| Shigella | Shiga | Intestine, kidney | Toxin has 5 subunits that bind to a |
| dysenteriae | | and brain | glycolipid receptor, Gb3, inducing |
| | | | endocytosis |
| Staphilococcus | enterotoxins | Intestines, T cells | Interacts with surface receptors inducing |
| aureus | | and macrophages | intracellular signaling cascade |
| Staphilococcus | Toxic shock | Vascular system | |
| aureus | syndrome | | |
| Escherichia coli | Labile toxin | Intestine (similar | Entry into host requires proteolytic |
| | | to cholera) | activation |
| Bordetella | Pertussis toxin | White blood cells | Toxin is composed of A and B oligomers. |
| pertusis | | | The B oligomer, containing five |
| | | | polypeptides, binds to cell receptors and |
| | | | delivers the S1 subunit. |
| Bordetella | Trachea toxin | Cilia bearing cells | |
| pertusis | | | |
| Bordetella | Adenylate | phagocytes | Internalized into macrophages via |
| pertusis | cyclase toxin | | CD11b/CD18 |
| Clostridium | Perfringens | Ileal Epithelial | |
| perfringens | enterotoxin | cells | |
| Clostridium | ToxinA/ToxinB | Intestine, immune | |
| difficile | | cells, neurons | |
| Clostridium | tetanus | neurons | |
| tetani | | | |

In certain cases, the targeting protein may be B subunit of Cholera toxin (CTB) (amino acid sequence as in GenBank Accession No. U25679; nucleotide sequence as in GenBank Accession No. U25679. Cholera toxin B subunit can be obtained from commercial sources such as Sigma, Invitrogen, List Biological Laboratories, Inc., Calbiochem, and the like. The CTB may be present as a pentamer (five B subunits; CtxB₅) in the complexes of the present disclosure. In certain cases, the CTB used in the complexes of the present disclosure may be a variant of a CTB that can form a pentamer.

The CTB containing complexes disclosed herein may be used to deliver a therapeutic agent to a mammalian target cell, such as, an intestinal cell (e.g., intestinal M cell or enterocyte) or a neuronal cell via binding of the CTB in the complex to a receptor (GM1 ganglioside) for CTB present on the target cell, such as, intestinal or neuronal cell.

In certain cases, the targeting protein may be an E. coli heat labile enterotoxin subunit B (LTB) (amino acid sequence of enterotoxin subunit B is as in GenBank Accession No. M17101; nucleotide sequence of enterotoxin subunit B is as inGenBank Accession No. M17101. The LTB may be present as a pentamer (five B subunits; LTB₅) in the complexes of the present disclosure.

In exemplary embodiments, the targeting protein may be a plant glycoprotein, such as lection, e.g., isolectin B4 (IB4) from Bandeiraea simplicifolia. IB4 binds mainly to terminal α-D-galactosyl residues on endothelial cells of blood vessels.

### Therapeutic Agents

A number of therapeutic molecules may be delivered to a target cell where activity of a therapeutic molecule is desired. Exemplary therapeutic agents include DNA (encoding a therapeutic protein, such as, insulin, dystrophin, erythropoietin, and the like), RNA (long double stranded RNA, siRNA, miRNA), peptide, proteins (e.g., therapeutic antibodies, such as chimeric or humanized antibodies) and the like. For example the therapeutic agent may be Infliximab (Remicade), Adalimumab (Humira), golimumab, 3F8, 8H9, Aducanumab, Cixutumumab, mAb/fragment antigen-binding (Fab) fragment, certolizumab pegol, Eldelumab, Etrolizumab, IMAB362, Natalizumab, Priliximab, AVX470, anti-α4β7 integrin antibody, analgesic; antidepressant; antidiabetic; antidiarrheal; antidiuretic; anti-epileptic; antifungal; antiglaucoma agent; antihemophilic; antihemorrhagic; antihistamine, antineoplastic, antineutropenic; antiproliferative; antiprostatic hypertrophy; antirheumatic; anti-ulcerative; antiviral; appetite suppressant; benign prostatic hyperplasia therapy agent; blood glucose regulator; bone resorption inhibitor; bronchodilator; carbonic anhydrase inhibitor; cardiac depressant; cardioprotectant; cardiotonic; cardiovascular agent; choleretic; cholinergic; cholinergic agonist; cholinesterase deactivator; cognition enhancer; depressant; dopaminergic agent; free oxygen radical scavenger; gastrointestinal motility effector; glucocorticoid; hair growth stimulant; prostate growth inhibitor; or radioactive agent.

In exemplary embodiments, the therapeutic agent may be a monoclonal antibody for treatment of IBD such as Crohn's disease and/or ulcerative colitis. Exemplary antibodies for treatment of IBD include: Adalimumab, Priliximab, Certolizumab pegol, Eldelumab, Fontolizuma, Infliximab, Natalizumab, Vedolizumab, and Visilizumab.

In certain embodiments, the DNA or RNA is not modified to include a functional moiety (e.g., a thiol group) that may form covalent linkage to a linker in a PEG-linker moiety under suitable conditions.

### Polyalkylene Glycol Linker Moiety

In certain embodiments, the complex for targeted delivery of a therapeutic agent to a target cell may include a polyalkylene glycol. For example, the polyalkylene glycol may be polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, derivatives thereof, combinations thereof, and the like. In certain embodiments, the polyalkylene glycol may be a polyethylene glycol (PEG). In certain cases, the polyalkylene glycol may include a linker moiety.

In certain embodiments, the complex for targeted delivery of a therapeutic agent to a target cell may include a polyalkylene glycol-linker moiety, where the polyalkylene glycol-linker moiety may be associated (via a non-covalent or covalent association) with a therapeutic agent to generate nanoparticles. The linker part of the polyalkylene glycol-linker in the nanoparticles then may be used to covalently attach the nanoparticles to the targeting protein.

Polyalkylene glycol-linker moiety may have the general structure polyalkylene glycol-X, where X is a linker moiety. In certain cases, the polyalkylene glycol-linker moiety may be a homobifunctional polyalkylene glycol having the general structure X-polyalkylene glycol-X. Exemplary homobifunctional polyalkylene glycol-linker moiety include: Sulfhydryl-reactive polyalkylene glycol Crosslinkers (e.g., bismaleimide-activated PEG compounds); and bis-succinimide ester-activated polyalkylene glycol compounds.

In certain cases, the polyalkylene glycol-linker moiety may be a heterobifunctional polyalkylene glycol having the general structure X-polyalkylene glycol-Y, wherein X and Y are different linkers. Exemplary heterobifunctional PEG-linker moiety include: HO-PEG-COOH: Hydroxyl PEG Carboxyl; HO-PEG-NHS: Hydroxyl PEG NHS Ester; HS-PEG-COOH: Thiol PEG Carboxyl; HS-PEG-SGA: Thiol PEG Succinimidyl Glutaramide; HO-PEG-NH2: Hydroxyl PEG Amine, HCl Salt; HS-PEG-NH2: Thiol PEG Amine, HCl Salt; NH2-PEG-COOH: Amine PEG Carboxyl, HCl Salt; TBOC-PEG-OH: t-Boc Amine PEG Hydroxyl; FMOC-PEG-OH: Fmoc Amine PEG Hydroxyl; TBOC-PEG-NH2: t-Boc Amine PEG Amine, HCl Salt; FMOC-PEG-NH2: Fmoc Amine PEG Amine, TFA Salt; TBOC-PEG-COOH: t-Boc Amine PEG Carboxyl; FMOC-PEG-COOH: Fmoc Amine PEG Carboxyl; TBOC-PEG-NHS: t-Boc Amine PEG NHS Ester; FMOC-PEG-NHS: Fmoc Amine PEG NHS Ester; ACLT-PEG-NHS: Acrylate PEG NHS Ester; MAL-PEG-OH: Maleimide PEG Hydroxyl; MAL-PEG-NH2: Maleimide PEG Amine; TFA Salt, MAL-PEG-COOH: Maleimide PEG Carboxyl; MAL-PEG-NHS: Maleimide PEG NHS Ester; BIOTIN-PEG-NHS: Biotin PEG NHS Ester; BIOTIN-PEG-MAL: Biotin PEG Maleimide; OPSS-PEG-NHS: OPSS PEG NHS Ester; AZIDE-PEG-NHS: Azide PEG NHS Ester; and AZIDE-PEG-NH2: Azide PEG Amine.

In certain embodiments, the PEG-linker moiety may be PEG-maleimide, e.g., MAL-PEG-OH: Maleimide PEG Hydroxyl; MAL-PEG-NH2: Maleimide PEG Amine; TFA Salt; MAL-PEG-COOH: Maleimide PEG Carboxyl; or MAL-PEG-NHS: Maleimide PEG NHS Ester.

In certain embodiments, the molecular weight of the PEG-linker moiety may be about 2 kDa to about 20 kDa, for example, 2 kDa, 3.5 kDa, 5 kDa, 7.5 kDa, 10 kDa, and 20 kDa, or may range between any two of the following values: 2 kDa, 3.5 kDa, 5 kDa, 7.5 kDa, 10 kDa, and 20 kDa.

In certain embodiments, the complex of the present disclosure does not include a cyclodextrin molecule such as, α-, β-, or γ-cyclodextrin.

In certain embodiments, the polyalkylene glycol-linker (e.g., PEG-linker) may be physically associated with a therapeutic agent via a non-covalent interaction, such as, electrostatic, π-effects, van der Waals forces, or hydrophobic interaction. In certain cases, the polyalkylene glycol-linker (e.g., PEG-linker) may be physically associated with a therapeutic agent via an electrostatic interaction, such as, ionic bonds. In certain cases, the polyalkylene glycol-linker (e.g., PEG-linker) may be physically associated with a therapeutic agent via covalent bond. In certain cases, the polyalkylene glycol-linker (e.g., PEG-linker) may be physically associated with a therapeutic agent via covalent and non-covalent interaction.

It is understood the complex of the present disclosure includes multiple complexes, for example a population of the complexes. A population of complexes may include heterogenous complexes or homogenous complexes. In certain embodiments, the complexes may be heterogenous and may include complexes that differ in the amount of therapeutic agent encapsulated in the complexes, for example.

### Method of Making Complexes

The complex of the present invention may be generated by performing two separate steps. In certain embodiments, the first step may include mixing the therapeutic agent with a polyalkylene-linker moiety.

In certain cases, the polyalkylene-linker moiety may be mixed at an amount of about 100 mg-200 mg, e.g., 100 mg, 110 mg, 120 mg, 130 mg, 140 mg, 150 mg, 170 mg, 180 mg, 200 mg and a range between any two of the following amounts: 100 mg, 110 mg, 120 mg, 130 mg, 140 mg, 150 mg, 170 mg, 180 mg, and 200 mg. In certain cases, the polyalkylene-linker moiety may be mixed with a therapeutic agent in a solution containing about 0.2 to 0.6M salt (e.g., NaCl, KCl, LiCl, ammonium salt (such as ammonium acetate), e.g., 0.2M, 0.3M, 0.4M, 0.5M, or 0.6M, and a range between any two of the following concentrations: 0.2M, 0.3M, 0.4M, 0.5M, and 0.6M. The polyalkylene-linker and/or the therapeutic moiety may be in water or saline. In certain cases, the polyalkylene may be PEG and the PEG-linker moiety may be PEG-maleimide.

In certain cases, the therapeutic reagent may be long dsRNA, siRNA or miRNA that may be mixed with the polyalkylene-linker moiety in an amount of about 2 μg-30 μg. In certain cases, the therapeutic reagent may be RNA, DNA, protein or peptide that may be mixed with a polyalkylene-linker moiety in an amount of about 1 μg-100 μg, e.g., 1 μg, 2 μg, 5 μg, 10 μg, 15 μg, 20 μg, 25 μg, 30 μg, 40 μg, 50 μg, 60 μg, 70 μg, 80 μg, 100 μg or 150 μg or at an amount that ranges between any two of the following amounts: 1 μg, 2 μg, 5 μg, 10 μg, 15 μg, 20 μg, 25 μg, 30 μg, 40 μg, 50 μg, 60 μg, 70 μg, 80 μg, 100 μg, and 150 μg. The amount of the therapeutic agent to be encapsulated in the complex of the present disclosure may be determined based on numerous factors, such as, desired pharmacological effect, effective dose of the therapeutic agent, the maximum tolerated dose, “MTD”, and the like.

In certain cases, the first mixing may be performed at room temperature (e.g. between 20° C. and 26° C., e.g., 23° C., 24° C., or 25° C.). In other embodiments, the mixing may be performed at 4° C. or at 37° C. or at a temperature in between 4° C. to 37° C. The mixing may be carried out for about 1 hour to about 48 hours, e.g., 1 hour, 2 hours, 4 hours, 6 hours, 10 hours, 12 hours, 16 hours, 20 hours, 24 hours, 36 hours, 48 hours or for a time in the range between any two of the following values: 1 hour, 2 hours, 4 hours, 6 hours, 10 hours, 12 hours, 16 hours, 20 hours, 24 hours, 36 hours, and 48 hours. In certain cases, the mixing may be carried out at room temperature for about 1-24 hours, e.g., 1-12 hours.

After the first mixing step is completed, the polyalkylene linker moiety is associated with the therapeutic agent to form nanoparticles. As noted herein, the association between polyalkylene-linker moiety and the therapeutic agent may be non-covalent or covalent. As used herein, the term “nanoparticles” refers to particles between 1 and 100 nanometers in size.

In certain embodiments, the nanoparticles are used in the next step of complex formation within 1 hour to 48 hours (e.g., within 12 hours, 16 hours, 24 hours, 36 hours, or 48 hours) after the nanoparticles are formed. The nanoparticles may be stored at 4° C. if they are not used immediately after they are made.

In certain embodiments, the nanoparticles are mixed with a targeting protein in a second step. The mixing may be carried out as above but under conditions that result in formation of covalent bonds between the targeting protein and the linker group on polyalkylene, thereby tethering the nanoparticles to the targeting protein, such as CTB. The targeting protein may be prepared in a buffer having a pH of about 6.5-7.5, for example, pH 6.5, 6.8, 7, 7.2, or 7.5, or a range between any two of the following values: pH 6.5, 6.8, 7, 7.2, and 7.5. In certain cases, a particular pH or pH range may be selected based on the therapeutic moiety in the nanoparticles. In certain cases, the polyalkylene-linker moiety-therapeutic protein nanoparticles may be mixed with the targeting protein at a pH of about 6.5-7.5, for example, pH 6.5, 6.8, 7, 7.2, or 7.5, or a range between any two of the following values: pH 6.5, 6.8, 7, 7.2, and 7.5. The covalent attachment of the polyalkylene-linker and nucleic acid (long dsRNA, siRNA or miRNA) nanoparticles may be carried out as above at a slightly acidic pH of about 6.5-6.8. In certain cases, the therapeutic reagent may be DNA, protein or polypeptide that may be mixed with a PEG-linker moiety in an amount of about 2 μg-30 μg. In certain embodiments, nanoparticles of PEG-linker moiety and protein or peptide may be incubated with the targeting protein at a neutral pH of about 7-7.2 or 7-7.5.

The targeting protein may be prepared in a high salt (e.g., NaCl, KCl) concentration (e.g. 5M to 0.8M) and mixed with the nanoparticles at a volume such that the resulting mixture has a salt concentration in the range of 0.2 to 0.6M salt (e.g., NaCl or KCl), e.g., 0.2M, 0.3M, 0.4M, 0.5M, or 0.6M, and a range between any two of the following concentrations: 0.2M, 0.3M, 0.4M, 0.5M, and 0.6M.

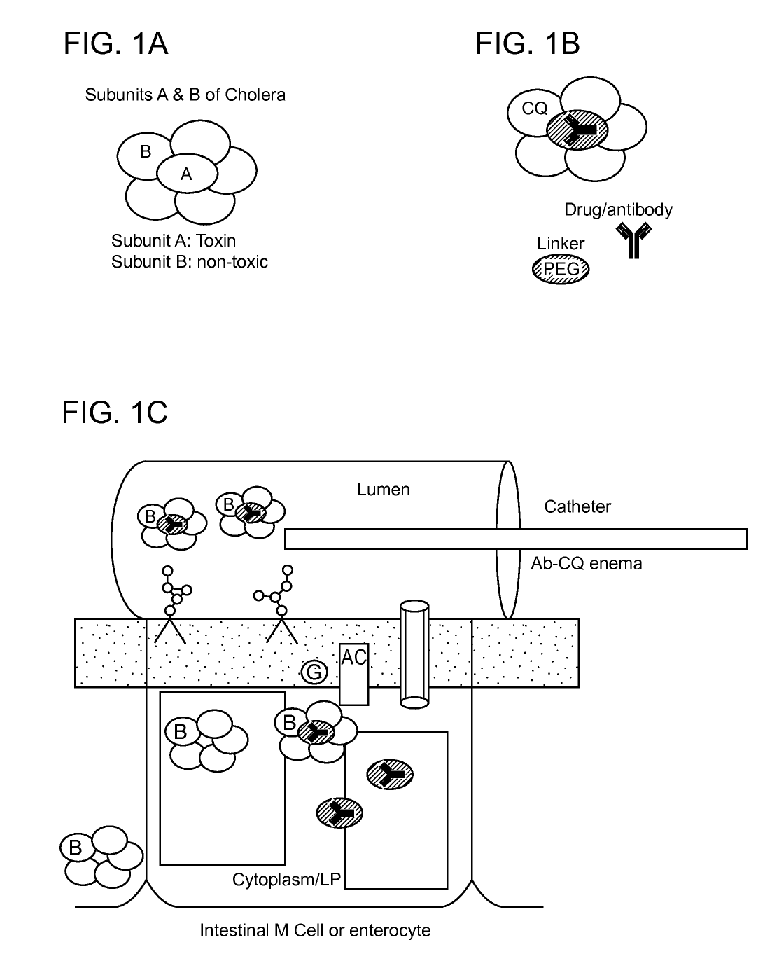

As described herein the present method of making the complex results in encapsulation of the nanoparticles in a targeting protein such that the targeting protein retains its native conformation. For example, in the methods of making the complex as described herein the CTB pentamer retains its pentameric structure, as depicted in FIG. 1B. As illustrated in FIG. 1C, a complex that includes a CTB pentamer with nanoparticles of PEG-linker-therapeutic agent (e.g., antibody), when administered locally to the intestine, is able to deliver the therapeutic agent to the intestinal cells by binding to GM1 receptor on intestinal cells—the complex is taken up by the intestinal cells by endocytosis, for example, resulting in the delivery of the complex into the cell.

Without wishing to be bound to theory, it is believed that the polyalkylene-linker moiety coats the therapeutic molecule and encapsulates it in step 1 to form nanoparticles. In step 2, the nanoparticles are covalently attached to the targeting protein via the linker moiety. However, the therapeutic molecule is not covalently linked to the targeting protein as the reactive groups (e.g. thiol moiety) of the therapeutic protein/peptide are not exposed and do not react to form a covalent bond with the targeting protein.

In certain cases, a purification step to isolate the nanoparticles and/or to separate out polyalkylene-linker moiety and therapeutic agent not associated into nanoparticles may be carried out prior to conjugation of the nanoparticles to a targeting protein. In certain cases, a purification step to isolate the complex of targeting protein and nanoparticles and/or to separate out polyalkylene-linker moiety and nanoparticles not covalently linked to form the complex may be carried out. Purification may be performed using steps standard in the field and may include chromatography, centrifugation, precipitation, differential solubilization, dialysis, ultrafiltration, electrophoresis, or a combination thereof.

### Pharmaceutical Compositions

The complexes of the present disclosure may be in the form of compositions suitable for administration to a subject. In general, such compositions are “pharmaceutical compositions” comprising the complexes and one or more pharmaceutically acceptable or physiologically acceptable diluents, carriers or excipients. In certain embodiments, the complexes are present in a therapeutically effective amount. The pharmaceutical compositions may be used in the methods of the present disclosure; thus, for example, the pharmaceutical compositions can be administered ex vivo or in vivo to a subject in order to practice the therapeutic and prophylactic methods and uses described herein.

The pharmaceutical compositions of the present disclosure can be formulated to be compatible with the intended method or route of administration; exemplary routes of administration are set forth herein. Furthermore, the pharmaceutical compositions may be used in combination with other therapeutically active agents or compounds (e.g., an FDA approved treatment) in order to treat or prevent the diseases, disorders and conditions as contemplated by the present disclosure.

The pharmaceutical compositions typically comprise a therapeutically effective amount of the complexes contemplated by the present disclosure and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that could be used in the pharmaceutical compositions and dosage forms. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments. Any drug delivery apparatus may be used to deliver the complexes, including implants (e.g., implantable pumps) and catheter systems, both of which are well known to the skilled artisan. Depot injections, which are generally administered subcutaneously or intramuscularly, may also be utilized to release the complexes disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, N.J.) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil may be employed including synthetic mono- or diglycerides. Moreover, fatty acids such as oleic acid find use in the preparation of injectables. Prolonged absorption of particular injectable formulations can be achieved by including an agent that delays absorption (e.g., aluminum monostearate or gelatin).

The pharmaceutical compositions containing the complexes of the present disclosure may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylenevinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods of preparing liposomes are described in, for example, U.S. Pat. Nos. 4,235,871, 4,501,728, and 4,837,028. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally-occurring gums, for example, gum acacia or gum tragacanth; naturally-occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

Formulations can also include carriers to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, may be employed.

The present disclosure contemplates the administration of the complexes in the form of suppositories for rectal administration of the drug. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

The complexes contemplated by the present disclosure may be in the form of any other suitable pharmaceutical composition (e.g., sprays for nasal or inhalation use) currently known or developed in the future.

The concentration of a complex disclosed herein in a formulation can vary widely (e.g., from less than about 0.1%, usually at or at least about 2% to as much as 20% to 50% or more by weight) and will usually be selected primarily based on fluid volumes, viscosities, and subject-based factors in accordance with, for example, the particular mode of administration selected.

### Method of Delivery

The present disclosure provides methods for treating or preventing a disease, such as, inflammatory bowel disease, neural pain, cancer, familial adenomatous polyposis (FAP) syndromes and the like, by the administration of the complexes, or compositions thereof, as described herein. Such methods may also have an advantageous effect on one or more symptoms associated with a disease, disorder or condition by, for example, decreasing the severity or the frequency of a symptom.

Where local delivery is desired, administration may involve administering the complex to a desired target tissue, such intestinal lumen, brain, spine, lung, heart, muscle, kidney, pancreas, liver, etc. For local delivery, the administration may be by injection or by placement of a composition containing the complex in the desired tissue or organ by surgery, for example. In certain cases, an implant, such as a catheter or a cannula implant that acts to deliver the complex at the site of implantation/insertion may be used. In some instances, a cannula or catheter may be inserted in a target tissue or organ.

In some instances, intracerebral, ventricular or intrathecal delivery protocols may be employed, e.g., as described in Buchli A D and Schwab M E (2005), Ann Med. 37:556-67; and Shoichet M S, Tator C H, Poon P, Kang C, Baumann M D (2007), Prog Brain Res. 161:385-92. In some instances, intranasal delivery protocols are employed, e.g., as described in Smith P F (2003), IDrugs. 6:1173-7; and Vyas T K, Tiwari S B, Amiji M M. (2006), Crit Rev Ther Drug Carrier Syst. 23:319-47.

In some embodiments, the complex may be formulated to cross the blood brain barrier (BBB). One strategy for drug delivery through the blood brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the composition of the complex disclosed herein when the compositions are administered by intravascular injection. Alternatively, drug delivery behind the BBB is by intrathecal delivery of therapeutics directly to the cranium, as through an Ommaya reservoir.

The present disclosure contemplates the administration of the disclosed complexes, and compositions thereof, in any appropriate manner. Suitable routes of administration include parenteral (e.g., intramuscular, intravenous, subcutaneous (e.g., injection or implant), intraperitoneal, intracisternal, intraarticular, intraperitoneal, intracerebral (intraparenchymal) and intracerebroventricular), oral, nasal, vaginal, sublingual, intraocular, rectal, topical (e.g., transdermal), sublingual and inhalation.

### Dosing

The complexes of the present disclosure may be administered to a subject in an amount that is dependent upon, for example, the goal of the administration (e.g., the degree of resolution desired); the age, weight, sex, and health and physical condition of the subject to be treated; the nature of the therapeutic molecule, and/or formulation being administered; the route of administration; and the nature of the disease, disorder, condition or symptom thereof (e.g., the severity of the disease and the stage of the disorder). The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (i.e., the maximum tolerated dose, “MTD”) and not less than an amount required to produce a measurable effect on the subject. Such amounts are determined by, for example, the pharmacokinetic and pharmacodynamic parameters associated with absorption, distribution, metabolism, and excretion (“ADME”), taking into consideration the route of administration and other factors.

An effective dose (ED) is the dose or amount of an agent that produces a therapeutic response or desired effect in some fraction of the subjects taking it. The “median effective dose” or ED50 of an agent is the dose or amount of an agent that produces a therapeutic response or desired effect in 50% of the population to which it is administered. Although the ED50 is commonly used as a measure of reasonable expectance of an agent's effect, it is not necessarily the dose that a clinician might deem appropriate taking into consideration all relevant factors. Thus, in some situations the effective amount is more than the calculated ED50, in other situations the effective amount is less than the calculated ED50, and in still other situations the effective amount is the same as the calculated ED50.

In addition, an effective dose of the complexes of the present disclosure may be an amount that, when administered in one or more doses to a subject, produces a desired result relative to a healthy subject. For example, an effective dose may be one that, when administered to a subject having IBD, achieves a desired reduction in inflammation of intestinal tissue relative to the inflammation present prior to start of the treatment by at least about 10%, at least about 20%, at least about 25%, at least about 30%, at least about 40%, at least about 50%, at least about 60%, at least about 70%, at least about 80%, or more than 80%.

An appropriate dosage level will generally be about 0.001 to 100 mg/kg of patient body weight per day, which can be administered in single or multiple doses. In some embodiments, the dosage level will be about 0.01 to about 25 mg/kg per day, and in other embodiments about 0.05 to about 10 mg/kg per day. A suitable dosage level may be about 0.01 to 25 mg/kg per day, about 0.05 to 10 mg/kg per day, or about 0.1 to 5 mg/kg per day. Within this range, the dosage may be 0.005 to 0.05, 0.05 to 0.5 or 0.5 to 5.0 mg/kg per day.

The dosage of the complexes of the present disclosure may be repeated at an appropriate frequency, which may be in the range of once per day to once every three months, depending on the pharmacokinetics of the complex or the therapeutic agent present in the complex (e.g. half-life) and the pharmacodynamic response (e.g. the duration of the therapeutic effect of the complex or the therapeutic agent present in the complex). In some embodiments, dosing is frequently repeated between once per week and once every 3 months. In other embodiments, complex may be administered approximately once per month.

In certain embodiments, the dosage of the disclosed complexes is contained in a “unit dosage form”. The phrase “unit dosage form” refers to physically discrete units, each unit containing a predetermined amount of complexes of the present disclosure, either alone or in combination with one or more additional agents, sufficient to produce the desired effect. It will be appreciated that the parameters of a unit dosage form will depend on the particular agent and the effect to be achieved.

### Patients

Subjects in need for treatment or prevention of a disease may be administered a complex of the present disclosure. The complex for administration to the patient may be selected on the basis of the disease that is to be treated or prevented. A subject that may benefit from administration of the complexes of the present disclosure may have or may be at risk of developing inflammatory bowel disease (IBD, including Crohn's disease (CD) and ulcerative colitis (UC)), neural pain (e.g., sciatic nerve pain), nerve injury, cancer (e.g., blood cancer, prostate cancer, colon cancer, brain cancer, familial adenomatous polyposis (FAP) syndromes, glaucoma, gastrointestinal motility disorders, and the like.

The subject may be a subject that has previously been treated with or is undergoing treatment for the disease using an alternate therapy. As such, in certain embodiments, the subject may be treated with a combination therapy.

In certain cases, the subject may have IBD (e.g., Crohn's disease or UC) and may be treated with a complex of the present disclosure where the targeting protein may be CTB or LTB or IB4 which may encapsulate a complex of PEG and a therapeutic agent for treating IBD. The therapeutic agent for treating IBD may be one or more of Infliximab (Remicade); Adalimumab (Humira), golimumab; mAb/fragment antigen-binding (Fab) fragment certolizumab pegol, Priliximab, Eldelumab, Fontolizuma, Infliximab, Natalizumab, Vedolizumab, Visilizumab, or AVX470.

In certain cases, a patient having or at risk of developing IBD may be administered a complex as disclosed herein by a systemic injection or by local delivery to the lumen of the intestine, for example, by using a catheter.

In certain cases, the subject may have neural pain, uncontrolled pain, or chronic pain. The subject may be treated by delivering neural cell signaling inhibitors to the neural cells via a complex provided herein. For example, a CTB may be used as the targeting protein for targeting the therapeutic agent to the neurons, e.g., neurons in a particular part of the spine or brain, e.g., by localized injection. In certain embodiments, the complex (targeting protein covalently linked to nanoparticles of polyalkylene-linker encapsulating a therapeutic molecule) may be placed in a pump and the complex can be delivered in multiple doses in a periodic manner (e.g., for treatment of pain in the spinal area). In other embodiments, the complex can be delivered to a subject via pH-sensitive capsule as oral delivery.

### Kits

The present disclosure also contemplates kits comprising the disclosed reagents for making the complexes of the present disclosure. The reagents may be as disclosed herein, such as, a PEG-linker moiety, a targeting protein, a therapeutic molecule, salt, buffer, and the like. The kits are generally in the form of a physical structure housing various components, as described below. Each component of the kit can be enclosed within an individual container and all of the various containers can be within a single package. A kit of the present disclosure can be designed for conditions necessary to properly maintain the components housed therein (e.g., refrigeration or freezing).

A kit may contain a label or packaging insert including identifying information for the components therein and instructions for their use. Labels or inserts can include manufacturer information such as lot numbers and expiration dates. The label or packaging insert may be, e.g., integrated into the physical structure housing the components, contained separately within the physical structure, or affixed to a component of the kit (e.g., an ampoule, tube or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via the internet, are provided.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Materials and Methods:

### Encapsulation:

Reagents:

CTB (obtained from Sigma, Catalog No. C9903; final concentration: 20 μg-50 μg in 0.1 M phosphate buffer), PEG-maleimide (‘PEG-Mal’ from Jenken Tech, Catalog No. A4011-5; final concentration: 120 mg-150 mg in water or saline); therapeutic molecule to be encapsulated (e.g., dsRNA/DNA/antibody/peptide/small organic molecule; concentration range (2-30 μg); sodium chloride (NaCl; final concentration 0.4M); phosphate buffer pH 7.0 for encapsulation of DNA/polypeptide/peptide; phosphate buffer pH 6.5-pH 6.8 (or slightly acidic) for RNA (e.g., dsRNA, siRNA).

Procedure:

Step 1, PEG-Mal was mixed with the therapeutic molecule to be encapsulated (dsRNA/DsDNA/antibody/peptide/small molecule) to the final concentration listed above in 0.4M NaCl to generate nanoparticles. Mixing was performed by tapping or gentle vortexing. Following the mixing step, nanoparticles were allowed to form at room temperature (RT) by incubating the mixture for duration of 2-4 hours.

Step 2, Add CTB (in 0.1M phosphate buffer, pH 7.0, except pH 6.8 for RNA encapsulation) to a desired volume of 5M NaCl (to achieve final concentration of 0.4M NaCl); incubate at RT for 1 h with gentle rocking or periodic shaking leading to formation of covalent bond between Mal and —SH moiety on CTB. The nanoparticles have been encapsulated within a CTB pentamer. The encapsulated nanoparticles are stored at 4° C. Notably, the therapeutic protein or nucleic acid is not covalently linked to CTB. Rather, the PEG-Mal that encapsulates the therapeutic protein or nucleic acid in the nanoparticles is covalently linked to CTB. Thus, when analyzed by gel electrophoresis, the complex is separated into CTB-PEG-Mal and the therapeutic molecule.

### Example 1: Cholera Toxin B Subunit Pentamer is Maintained Upon Incubation with PEG-Mal

CTB has been covalently linked to molecules such as proteins or DNA in order to target the protein or DNA to a target cell. One of the reagents frequently used for covalently linking DNA/protein to CTB is a heterobifunctional crosslinking reagent succinimidyl-4-(N-maleimidomethyl)cyclohexane-1-carboxylate (SMCC). An alternate reagent, PEG-Mal is presented here.

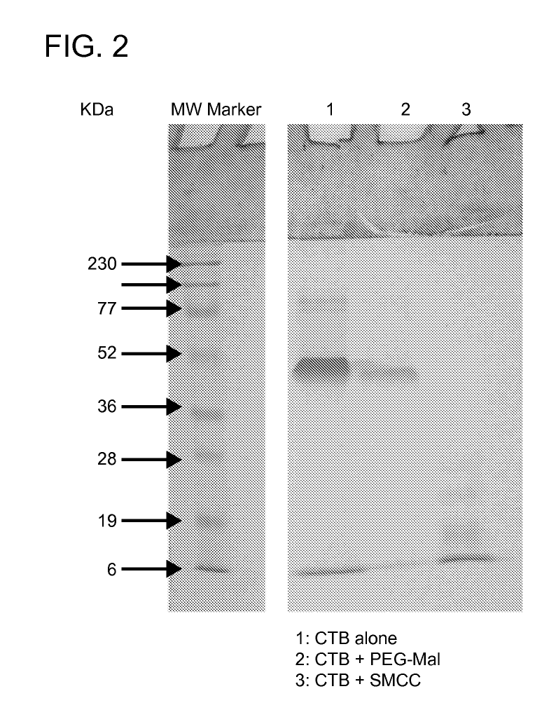

FIG. 2 depicts migration of CTB, CTB-SMCC and CTB-Mal-PEG in a native gel. CTB (Sigma; Catalog No. C9903) is present as a pentamer (see FIG. 2, lane 1). However, upon conjugation with SMCC, the pentameric structure of CTB breaks down and monomers, dimers, and trimers of CTB are generated (FIG. 2, lane 3). In contrast, when CTB is complexed with PEG-Mal, the pentameric structure of CTB is retained (FIG. 2, lane 2). In these experiments, a therapeutic molecule was not included.

The retention of the CTB pentamer as compared to the formation of CTB monomers, dimers, or trimers is important for the use of the CTB to encapsulate nanoparticles containing a molecule of interest (e.g. a therapeutic agent) for the purpose of delivering the encapsulated nanoparticle to a target cell while invoking a minimal immune response. In contrast, the non-pentameric CTB in the CTB-SMCC-DNA/protein complex is less likely to encapsulate and deliver a molecule of interest to the target cell and is more likely to invoke an immune response to the molecule of interest. Without being bound to a particular theory or mechanism, it is believed that the retention of the pentameric structure facilitates optimal encapsulation of the molecule of interest to be delivered to the target cell and proper delivery into the target cell.

Figure. 2. Incubation of CTB with PEG-Mal in 0.4M NaCl, pH 7.0 retains its pentameric structure, whereas, incubation of CTB with SMCC results in “breakdown” of CTB pentamers to mono-, di-, or tri-mers.

### Example 2: Anti-TNF-α Antibody Encapsulated in Cholera Toxin B Subunit

Inflammatory Bowel Disease (IBD) is currently treated with anti-TNF-α antibodies such as, Humira and Remicade. However, these treatments suffer from side-effects. It is proposed that targeting the anti-TNF-α antibodies to the site of action by using a targeting moiety that specifically binds to cells in the intestine, would decrease side effects and will further enhance efficacy by delivering the antibody to the target cells.

A rat mouse anti-TNF-α antibody (BioXCell, Cat No. BE0058, clone # XT3.11) was non-covalently complexed with PEG-Mal and the resulting nanoparticles encapsulated in CTB as outlined in materials and methods. PEG-Mal encapsulated in CTB without the rat anti-TNF-α antibody served as a negative control. PEG-Mal encapsulated in CTB is referred herein as Complex Q (CQ). The anti-TNF-α antibody and PEG-Mal nanoparticles encapsulated in CTB are referred to as CQ-TNF-α.

Crohn's Model:

To generate Crohn's colitis in rats or mice a commonly accepted model in which an enema of trinitrobenzene sulfonic acid (TNBS) induces an immunologically mediated colitis was used. After the mucosa is first made permeable with ethanol, TNBS serves as a hapten that is covalently bound to self molecules, inducing a delayed-type hypersensitivity response that develops into colitis. Granulomas, together with infiltration of inflammatory cells, become visible in all layers of the intestine. The isolated macrophages produce large amounts of IL-12, and lymphocytes produce significantly high concentrations of IFN-γ and IL-2. This evidence suggests that colitis in this model is induced by a T-helper type-1 response and is an excellent Crohn's disease model, both in its mechanism and its end effects.

The trinitrobenzene sulfonic acid (TNBS)-induced rat and/or mice model of Crohn's colitis was used to assess therapeutic efficacy of CQ-TNF-α and TNF-α. CQ served as the negative control. CQ-TNF-α, TNF-α or CQ was administered to rats by enema. Rats were administered 3 mg/Kg of TNF-α antibody present in CQ (Q is CTB-PEG-Mal). This dose of TNF-α antibody is well below the dose at which TNF-α antibody is usually administered (usual dose is 25 mg/Kg).

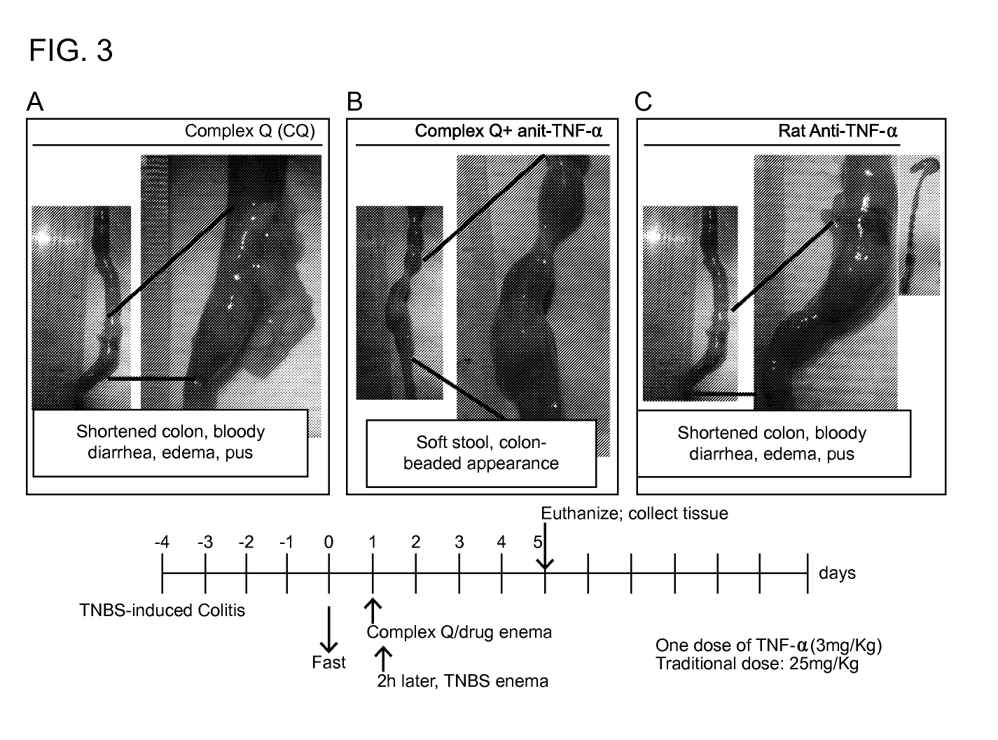

Colon of the rats was examined after administration of CQ-TNF-α (3 mg/Kg), TNF-α (3 mg/Kg), or CQ (25 μg dose). Visual inspection revealed vastly improved colon in rats treated with CQ-TNF-α. The effect of CQ-TNF-α was superior to that observed with TNF-α and CQ. (See FIG. 3). The data presented in FIG. 3 demonstrates that when complexed in CQ, even a lower dose of anti-TNF-α antibody is effective. Importantly the antibody is undetectable in serum, which implies that most of the antibody is stably encapsulated CQ-TNF-α, which would increase delivery to target cells and decrease side effects from off target delivery of the antibody.

FIGS. 3A-3C. Anti-TNF-α when delivered locally with Complex Q, is effective in decreasing inflammation (FIG. 3B). Inset shows normal rat colon. Experimental time line for delivery of therapeutic moiety in Crohn's model is shown.

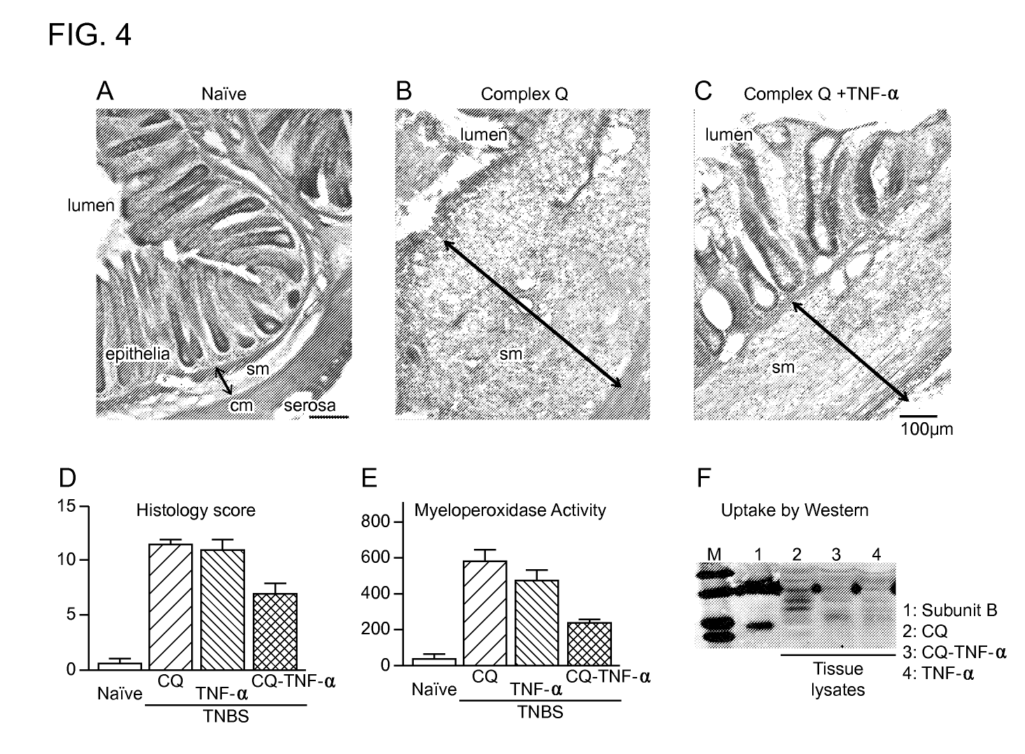

FIG. 4A-4F shows that Complex Q-anti-TNF decreases histological damage (FIGS. 4A-4D), including decrease in inflammatory cell infiltrate as assessed by decrease in myeloperoxidase (MPO) activity (FIG. 4E). CQ can be detected in the colon tissue within 4 h of injection (FIG. 4F, lane 2) and is undetectable within 24 h to 3 days (FIG. 4F, lane 3 and not shown). Lane 1: positive control, lane 4: negative control.

### Example 3: Analysis of Local Delivery of Complex Q-Anti-TNF-α in Mice Colon

Based on the findings in the above rat study, a parallel study in mice was designed to determine whether local delivery of Complex Q-anti-TNF-α results in targeted delivery to the colon cells or Complex Q-anti-TNF-α can spread to other tissues such as the liver and the kidney and is also detected in the serum.

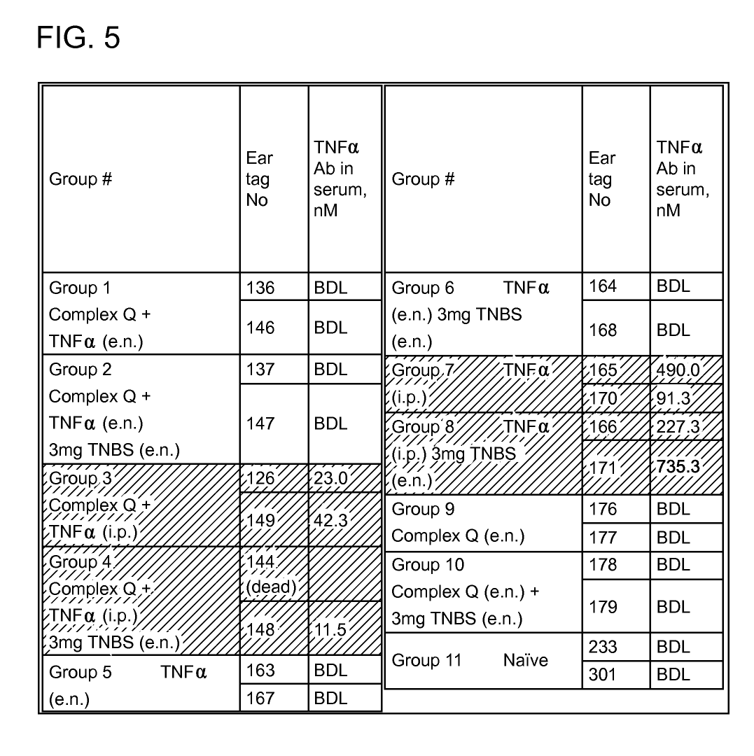

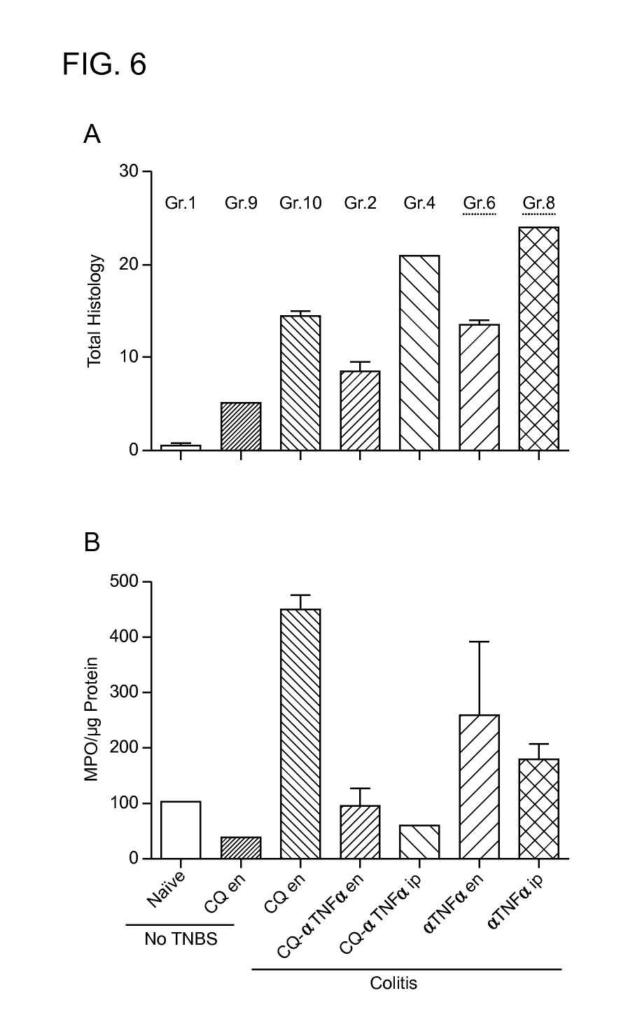

Anti-TNF-α antibody in Complex Q was administered either locally in the colon as an enema, or injected intraperitoneally (ip) for systemic delivery. FIG. 5 delineates control and experimental groups for a follow-up experiment to detect TNF-α by ELISA. The highlights of the results were as follows: 1) As expected, anti-TNF-α, when given by traditional IP route (Group 8 in FIG. 5), could be detected both in the serum and in colon homogenates (FIG. 5 and not shown). 2) As predicted, anti-TNF-α, when delivered via CQ enema, was not detected in the serum samples or in colon homogenates (below detection level; BDL). However, as predicted, histopathology of gut tissue of mice in Group 2 was better (FIG. 6A) than in mice with colitis and no treatment (Group 10) and mice that received ip anti-TNF-α (Group 8). 3) Importantly, MPO activity, a surrogate marker for neutrophil infiltration in the inflamed gut, was significantly decreased in Group 2 vs. Groups 8 &10 (FIG. 6B). These new data suggest that the local delivery platform is effective.

FIG. 5 provides results from detection of anti-TNF-α antibody in serum. BDL=below detection level; e.n.=enema; i.p.=intraperitoneal

FIGS. 6A and 6B provides a histological and biochemical analysis of colon of mice in the indicated treatment/control group.

### Example 4: Targeted and Local Delivery of Humira Encapsulated in Cholera Toxin B Subunit

A dose of 3-5 mg/kg (mice) and 15 mg/Kg (in rats) of TNBS in 50% EtOH is effective in initiating an inflammatory response and results in colitis lasting over 2 weeks in mice and rats. Before TNBS colitis was induced, mice were fasted overnight with free access to water. Mice were anesthetized with isoflurane (˜3%, O₂ ˜0.8-1 mmHg). The free tip of a catheter (lightly lubricated with Vaseline) attached to a 1 ml syringe (containing TNBS solution) was gently inserted into the anus (4 cm up in mice and 8 cm in rats). Colitis was induced by infusing 50 μl of TNBS solution (200 μl in rats) into colon via the catheter attached to a 1 ml syringe. The anus was pinched around the catheter to prevent leakage throughout infusion and the pelvis was kept elevated for about 2 minutes after catheter removal. For groups of mice receiving CQ-Humira, first, the complex was given via an enema as described above, followed by an enema of TNBS to induce colitis. In groups of mice, Humira was injected intraperitoneally and colitis was induced as above. A group of mice were given TNBS enema to induce colitis. Controls: Mice were given a saline enema or naïve animals with no treatment served as additional controls.

The TNBS-induced Crohn's colitis model in mice was used to assess the efficacy of Humira complexed with PEG-Mal and encapsulated in CTB (Humira-CQ) as indicated in materials and methods.

Although the standard dose of Humira in mice/rodents is about 25 mg/Kg for intraperitoneal administration, 1 mg/Kg dose of Humira in CQ complex or of the uncomplexed antibody was administered intraperitoneally. Humira-CQ or Humira was administered 3 days after administration of TNBS to induce Crohn's colitis.

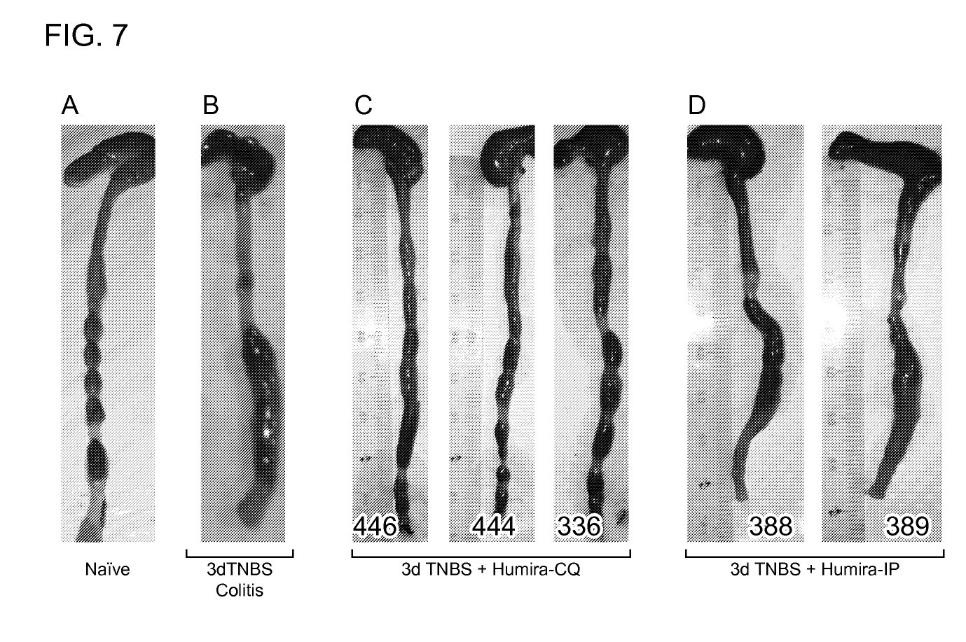

Colon biopsy samples from mice are shown in FIG. 7A-7D. A colon from a normal rat is depicted in FIG. 7A; colon of a mice administered TNBS is shown in FIG. 7B; colon of a rats (#446, #444, #336) administered TNBS and treated with Humira-CQ 3 days after administration of TNBS is shown in FIG. 7C; colon of mice (#388, and #389) administered TNBS and treated with IP injection of Humira is shown in FIG. 7D. The colons were analyzed on day 3 after treatment with Humira.

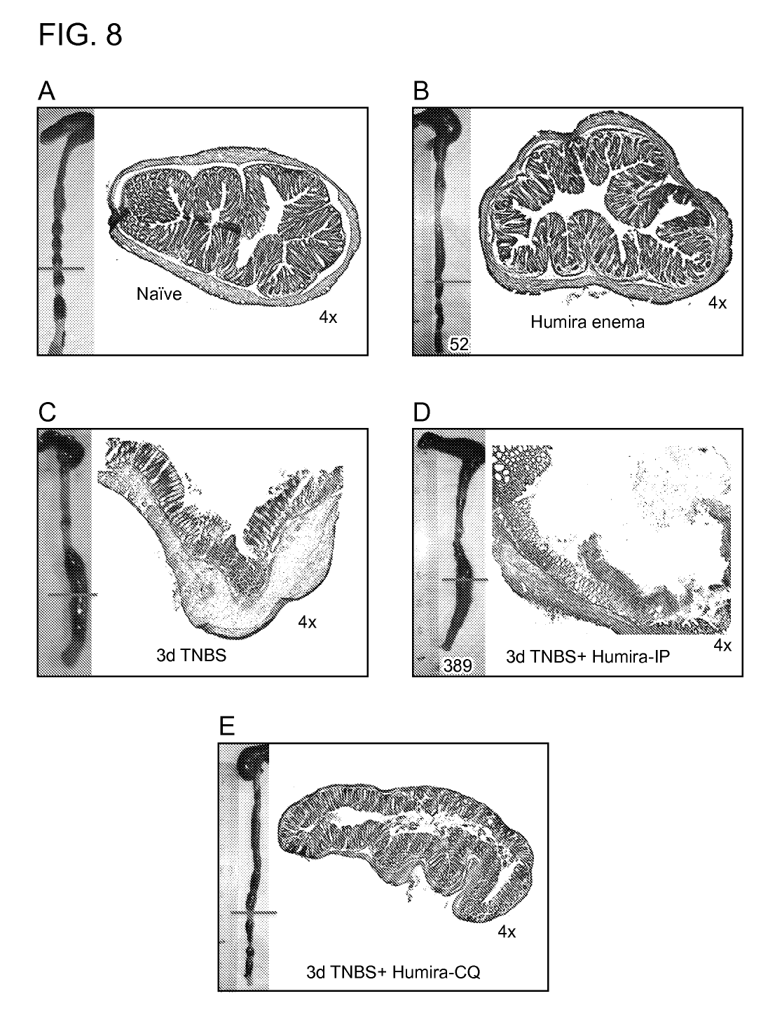

FIGS. 8A-8E provides a comparison of the effect of the route of administration of the effectiveness of treatment. Colitis was induced by a rectal enema of a bolus of 3-5 mg/Kg TNBS in mice. Humira given via IP route at a lower dose is not effective in ameliorating gross inflammation (FIG. 8D) as assessed by examination of H&E stained colon sections from areas of colon (shown by a red line), whereas Humira-CQ given as a rectal enema was effective in ameliorating inflammation (FIG. 8E) compared to TNBS (colitis) alone (FIG. 8C). Naïve mice given Humira alone as a rectal enema served as controls.

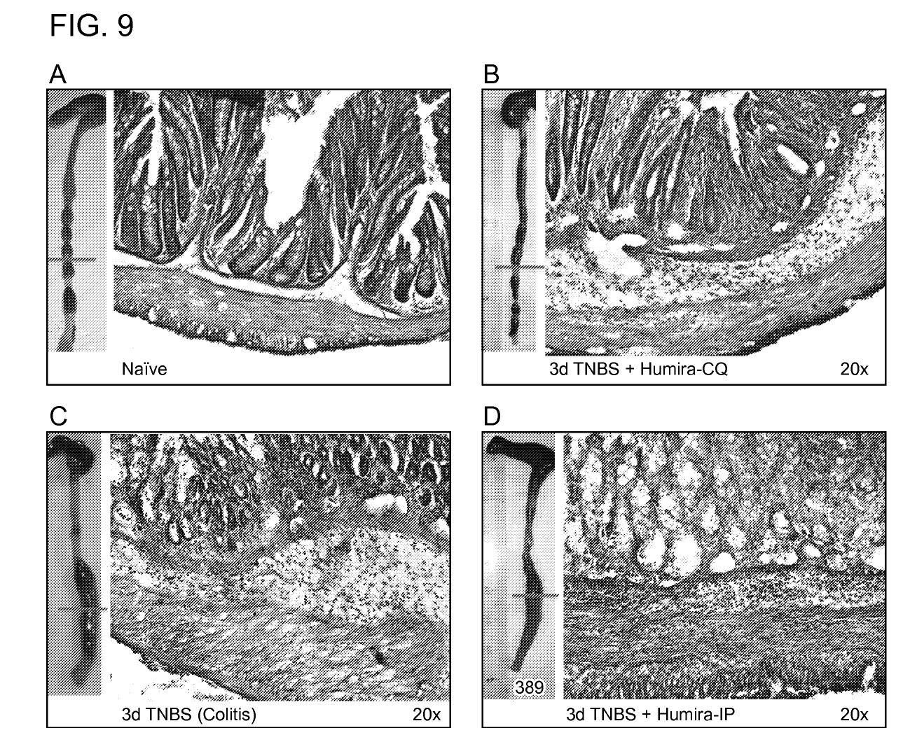

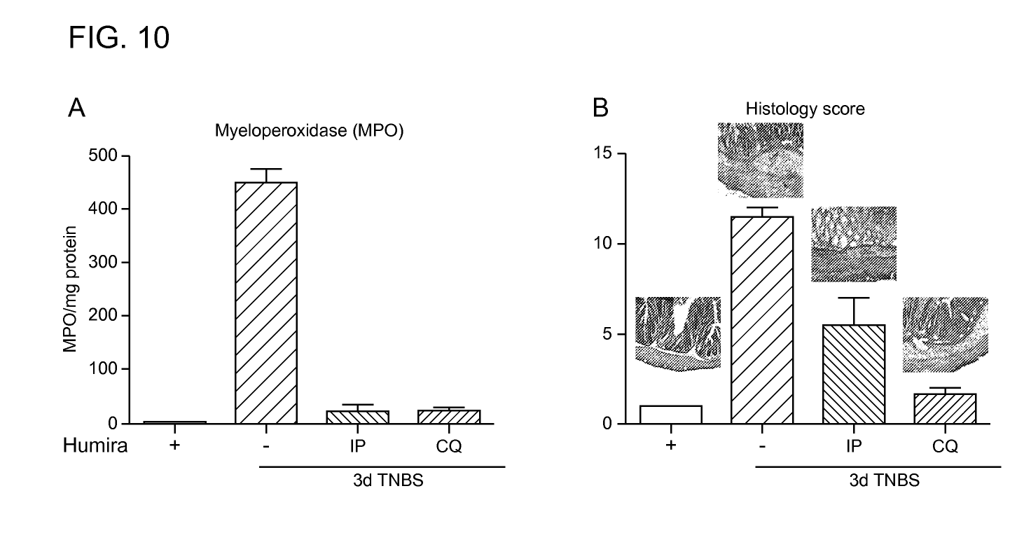

FIGS. 9A-9D provides a higher magnification image of the tissue depicted in FIG. 8. FIGS. 10A-10B depict reduced myeloperoxidase (MPO) activity from mice treated with Humira-CQ or Humira. Neutrophil infiltration at the site of inflammation is a hallmark of Crohn's disease. MPO activity of these infiltrated neutrophils is increased during inflammation. As expected, induction of colitis with TNBS increased MPO activity in colonic homogenates from mice. CQ-Humira and ip administration of Humira was equally effective in reducing MPO activity during colitis from colonic lysates of mice (FIG. 10A). Next, a pathologist scored the H&E-stained colonic slides for changes in edema, necrosis, and infiltration of immune cells. TNBS-colitis increased the total histological score (FIG. 10B). CQ-Humira was very effective in decreasing inflammation, with histological damage being similar to those of controls (mice with saline enema or naïve mice with no insult). IP administration of Humira was not as effective as our formulation in decreasing total histological damage (FIG. 10B). Taken together, our data suggests that the biological activity of the encapsulated Humira (CQ-Humira) is similar to that of the unmodified Humira as both are equally effective in reducing the MPO activity, however, CQ-Humira is more effective in reducing total histological damage than Humira given via the ip route.

### Example 5: Targeted Delivery of Remicade Encapsulated in Cholera Toxin B Subunit

The TNBS-induced mouse Crohn's colitis model was used to assess the efficacy of Remicade complexed with PEG-Mal and encapsulated in CTB (Remicade-CQ) as described in materials and methods.

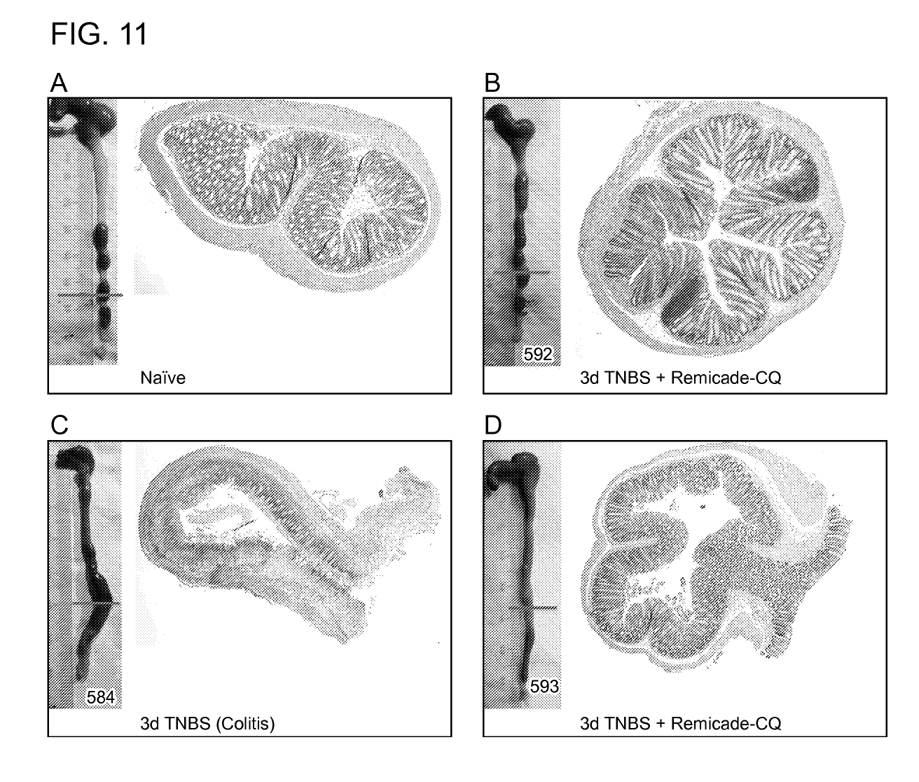

As shown in FIGS. 11A-11D, Remicade-CQ significantly decreased inflammation in the intestine of the mice. TNBS-colitis increased the total histological score (FIG. 11C) as assessed by gross examination of the H&E sections of the colons (red line depicts the area of colons collected for histological analysis). CQ-Remicade was very effective in decreasing inflammation (FIG. 11B,D), with histological damage being similar to those of controls (mice with saline enema or naïve mice with no insult).

### Example 6: Targeted Delivery of Urocortin 1 (Ucn-1) Encapsulated in Cholera Toxin B Subunit

Next, we tested delivery of a small peptide (˜38 aa). 5FAM-labeled ucn1 peptide was synthesized and 30 μg of the labeled-peptide was encapsulated with PEG-Mal to produce nanoparticles and subsequently the nanoparticles were covalently attached to CTB using the procedure described in the materials and methods. CQ-Ucn1 was administered via a rectal enema as described in Example 4 followed by a bolus of TNBS enema in groups of mice. We have previously shown that Ucn1 is induced after a bolus of TNBS (Chang et al AJP, 2011) and that exogenous administration of Ucn1 reduces colitis-induced inflammation in rats and mice.

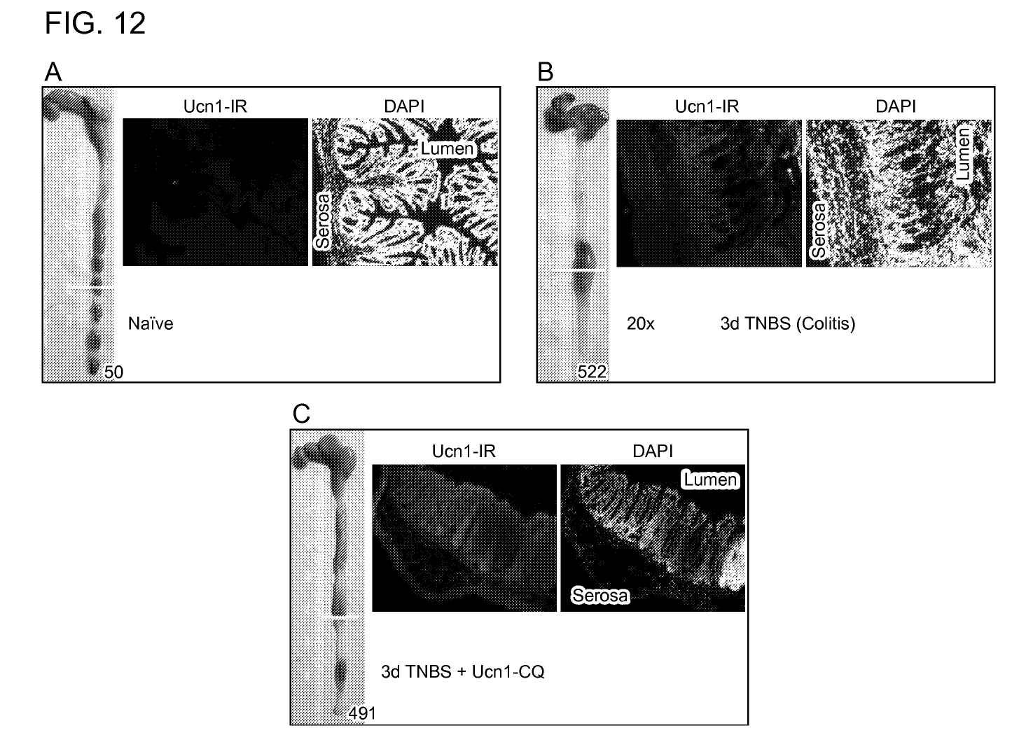

Levels of immunoreactive Ucn1 (Ucn1-IR) were determined by incubating colon sections with a primary Ucn1 antibody, followed by detection with RRX-labeled secondary antibody. Very low levels of Ucn1-IR were present in the colon tissue of naïve mice (FIG. 12A). Colitis induced Ucn1-IR in the colon (FIG. 12B), whereas mice that were given CQ-Ucn1 show robust Ucn1-IR in the colons and show reduced inflammation, suggesting efficient uptake of the encapsulated Ucn1.

### Example 7: Analysis of CQ Complex Containing dsRNA

dsRNAs for Corticotropin-releasing factor receptor1 (dsCRF1) and purinergic receptor P2X3R (dsP2X3) were first incubated with PEG-Mal as described in Materials and Methods, followed by incubation with CTB, allowing the linker (PEG) to form covalent bonds with CTB, thereby changing the physical structure and properties of CTB.

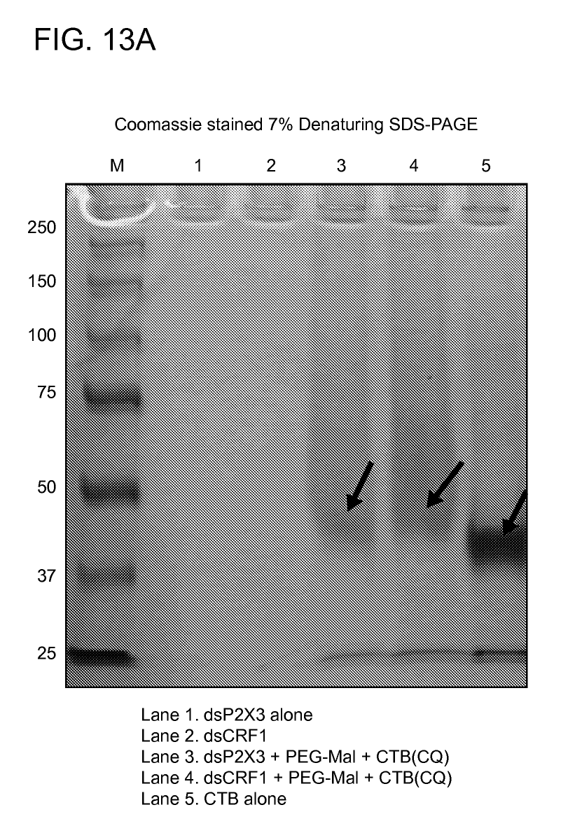

dsRNAs alone; or in CQ; and CTB alone was then separated on a 7% SDS-PAGE and stained with Coomassie blue to detect proteins (FIG. 13A). FIG. 13A, lanes 1 & 2 had dsRNA alone that is not stained by Coomassie stain; lanes 3 & 4 show that the dsRNA-CQ at higher molecular weight than CTB alone.

dsRNAs alone or in CQ and CTB alone was then separated on a 7% non-denaturing/native PAGE and stained with Coomassie blue to detect proteins (FIG. 13B) or ethidium bromide to detect dsRNA (FIG. 13C). The non-denaturing gel allows for the complex to separate based on its folded structure and mass. FIGS. 13B and 13C, lanes 1 & 2 had dsRNA alone that is not stained by Coomassie stain; lanes 3 shows that the dsRNA-CQ at higher molecular weight than CTB alone (FIG. 13C). Since the dsRNA is not covalently linked to the PEG-Mal or CTB, under an electric field, it falls apart from the complex and is separated based on its mass and structure (FIG. 13C).

### Example 8: Analysis of CQ Complex Containing Anti-TNF-α Antibody

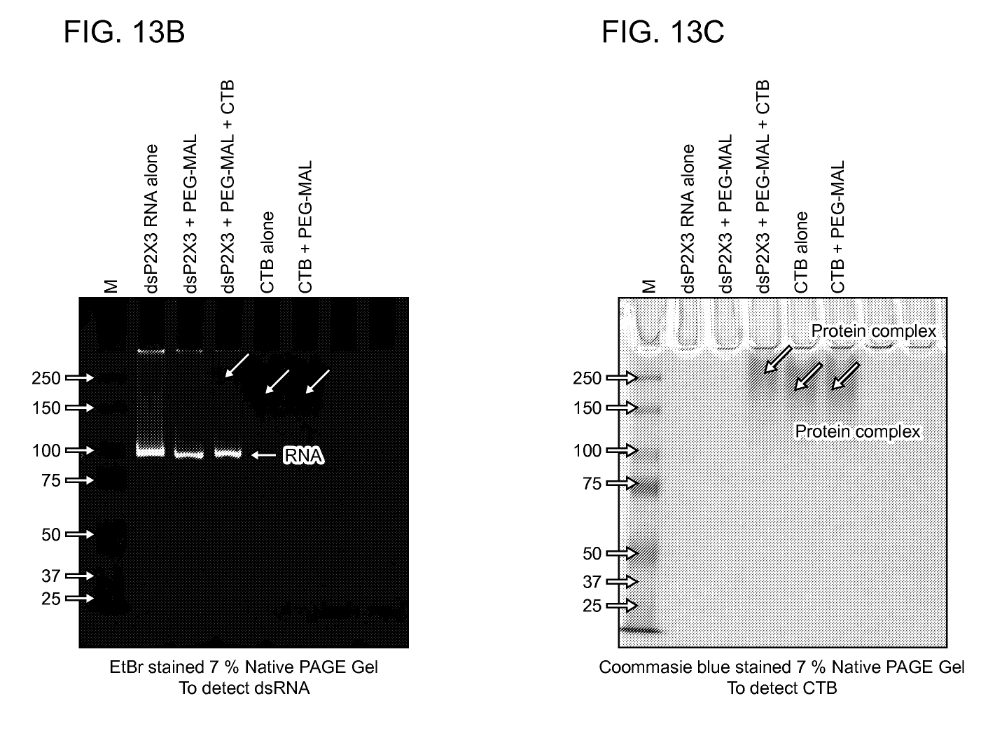

Anti-TNF-α antibodies (Humira and Remicaid) were first incubated with PEG-Mal as described in Materials and Methods, followed by incubation with CTB, allowing the linker (PEG) to form covalent bonds with CTB, thereby changing the physical structure and properties of CTB. To determine if incubation of CTB with PEG-Mal (reverse order) first followed by addition of anti-TNF-α (Humira or rat monoclonal anti-TNF-α) would result in a different complex than if the antibody is first incubated with PEG-Mal and then incubated with CTB, both kinds of complexes were separated on a 7% non-denaturing/native PAGE and stained with Coomassie blue to detect proteins (FIG. 14). The non-denaturing gel allows for the complex to separate based on its folded structure and mass. If the therapeutic antibody is first incubated with PEG-Mal, followed by incubation with CTB, then CTB does not covalently bind to the therapeutic antibodies (FIG. 14, lanes 2 and 8), as under an electric field, CTB falls apart from the complex and is separated based on its mass and structure (runs below Humira or Remicaid). However, if the reaction order is reversed, allowing for random covalent linkage between CTB and PEG-Mal, or CTB and the antibody, then the complex has a different mass/structure (FIG. 14, lanes 3 & 9) and is separated at a different size than antibody-CQ (compare FIG. 14, lanes 2 & 8 vs 3 & 9). Thus it is evident that the therapeutic antibody does not get covalently linked to CTB (if proper reaction order is followed). These data show that the therapeutic agent need not be covalently linked to CTB and further, that even in absence of a covalent attachment of the therapeutic agent to CTB, the complex is stable and active (see data for activity, provided herein).

### Example 9: Immunogenicity Studies

About 40% of the patients on anti-TNF-α therapy become refractile to this treatment after repeat dosing due to development of antibodies to anti-TNF-α, resulting in reduced bioavailability of the therapeutic antibody. CTB, a component of our carrier platform and CQ is a known immunogen when given systemically. It was reasoned that the route of administration is key and when given as an enema, even after repeat dosing, we would see minimal immune response, with low titer for IgG against CTB in serum of mice given a CQ enema vs intraperitoneal (ip) dosing.

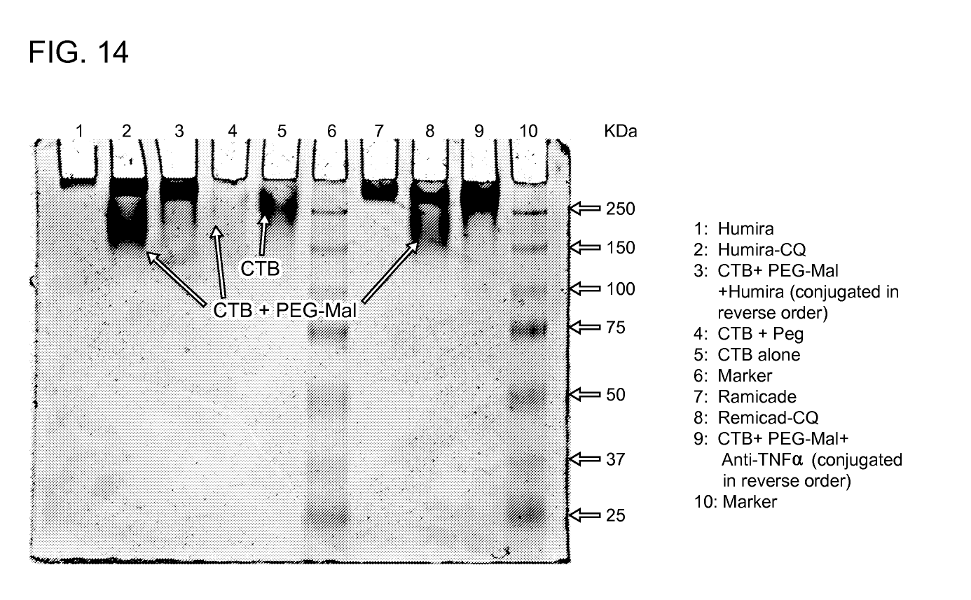

Three (3) doses of CQ (2 weeks apart) were administered in mice and an equivalent amount of CTB alone (no complex Q) was given ip, as control (FIG. 15). Naïve mice served as an additional control group. A pre-immune serum was collected before dosing (see attached design) and 2 weeks after each dose.

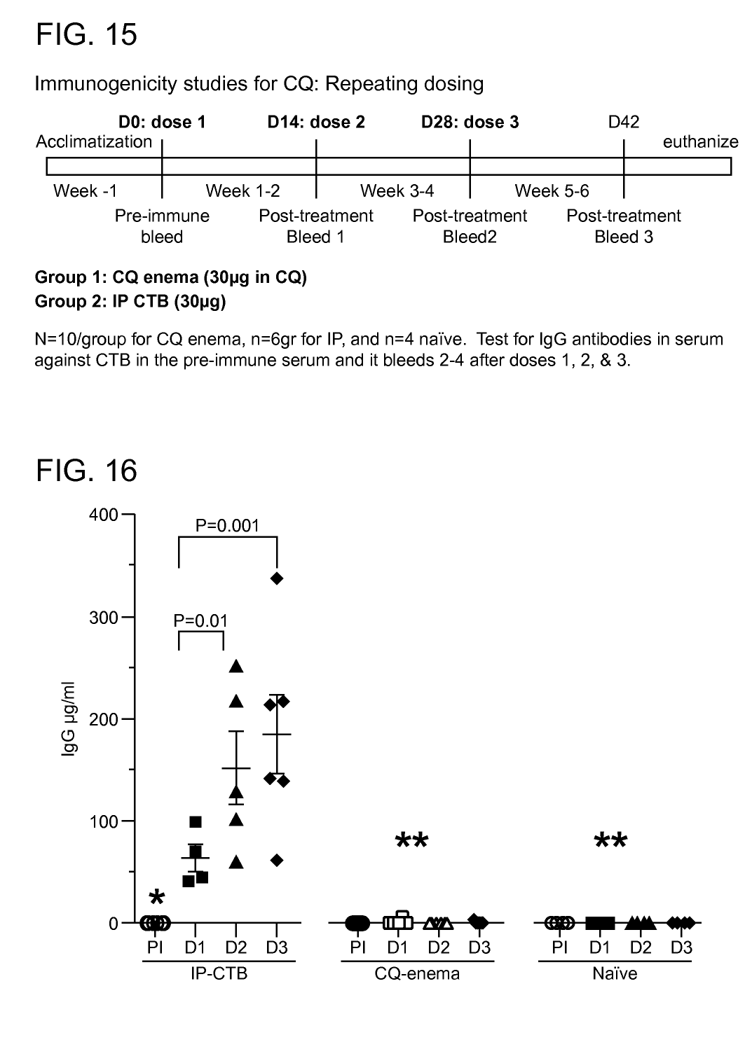

As seen in FIG. 16, no detectable IgG (using ELISA) is present in serum from uninjected naïve mice, or in pre-immune bleed. The titer of IgG increases after a 2^{nd} ip dose of CTB that is statistically significant (p<0.05) from bleed 1. Importantly, even after 3 doses of CQ enema, no appreciable increase was observed in IgG titer in serum of mice in bleeds 1-3. These data shows that the route of delivery is important and CQ enema does not result in accumulation of appreciable amounts of anti-CTB antibodies in circulation, thereby minimizing side-effects.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A complex comprising:
nanoparticles comprising a therapeutic agent associated with a polyalkylene glycol molecule, the polyalkylene glycol molecule comprising a linker moiety; and
a targeting protein covalently linked to the nanoparticles via the linker moiety.

2. The complex of claim 1, wherein the targeting protein is a bacterial protein that specifically binds to a target mammalian cell.

3. The complex of claim 2, wherein the bacterial protein forms a bacterial toxin complex.

4. The complex of claim 3, wherein the bacterial protein is a pentamer of cholera toxin subunit B.

5. The complex of claim 1, wherein the targeting protein is a plant protein that specifically binds to a target mammalian cell.

6. The complex of claim 5, wherein the plant protein is a glycoprotein.

7. The complex of claim 6, wherein the glycoprotein is a lectin.

8. The complex of claim 6, wherein the glycoprotein is multimer of isolectin B4.

9. The complex of any one of claims 1-8, wherein the therapeutic agent is selected from a group consisting of deoxyribonucleic acid (DNA), ribonucleic acid (RNA), peptide, and protein.

10. The complex of claim 9, wherein the protein is an antibody.

11. The complex of claim 9, wherein the DNA encodes a therapeutic protein.

12. The complex of claim 9, wherein the RNA is short inhibitory RNA (siRNA).

13. The complex of claim 9, wherein the RNA is microRNA (miRNA).

14. A method of making a complex comprising:
nanoparticles comprising:
a therapeutic agent associated with a polyalkylene glycol molecule, the polyalkylene glycol molecule comprising a linker moiety;
a targeting protein covalently linked to the nanoparticles via the linker moiety,
the method comprising:
mixing the polyalkylene glycol molecule with the therapeutic agent under conditions that result in association of polyalkylene glycol molecule with the therapeutic agent to generate the nanoparticles;
mixing the nanoparticles with the targeting protein under conditions that result in covalent association of the nanoparticles to the targeting protein via the linker moiety.

15. The method of claim 14, wherein the first mixing step is carried out for a period of 1 hr to 24 hrs.

16. The method of any one of claim 14 or 15, wherein the second mixing step is carried out at room temperature for a period of 1 hr to 24 hrs.

17. The method of any one of claims 14-16, wherein the first mixing step is carried out at room temperature.

18. The method of any one of claims 14-16, wherein the second mixing step is carried out at room temperature.

19. A method of delivering a therapeutic molecule to a target cell comprising:
administering a complex to a subject, wherein said administration is by localized administration, the complex comprising:
nanoparticles comprising a therapeutic agent associated with a polyalkylene glycol molecule, the polyalkylene glycol molecule comprising a linker moiety; and
a targeting protein covalently linked to the nanoparticles via the linker moiety,
wherein the targeting protein specifically binds to the target cell, and
wherein binding of the targeting protein to the target cell results in uptake of the complex by the target cell.

20. The method of any one of claims 14-19, wherein the targeting protein is a bacterial protein that specifically binds to a target mammalian cell.

21. The method of claim 20, wherein the bacterial protein forms a bacterial toxin complex.

22. The method of claim 21, wherein the bacterial protein is a pentamer of cholera toxin subunit B.

23. The method of any one of claims 14-19, wherein the targeting protein is a plant protein that specifically binds to a target mammalian cell.

24. The method of claim 23, wherein the plant protein is a glycoprotein.

25. The method of claim 24, wherein the glycoprotein is a lectin.

26. The method of claim 6, wherein the glycoprotein is multimer of isolectin B4.

27. The method of any one of claims 1-8, wherein the therapeutic agent is selected from a group consisting of deoxyribonucleic acid (DNA), ribonucleic acid (RNA), peptide, and protein.

28. The method of claim 27, wherein the protein is an antibody.

29. The method of claim 27, wherein the DNA encodes a therapeutic protein.

30. The method of claim 27, wherein the RNA is short inhibitory RNA (siRNA).

31. The method of claim 27, wherein the RNA is microRNA (miRNA).
